(19) United States
(12) Patent Application Publication
(10) Pub. No.: US 2007/0098810 A1
(43) Pub. Date: May 3, 2007

(54) Preparation for solid calcium bolus product

(76) Inventor: John H. Lee, Olathe, KS (US)

Correspondence Address: RIGEL TECHNOLOGY CORPORATION, 10677 WIDMER, LENEXA, KS, 66215, US

(21) Appl. No.: 11/263,608

(22) Filed: Nov. 1, 2005

(51) Int. Cl. A61K 31/59; A61K 33/42; A61K 33/10; A61K 33/08; A01N 45/00; A01N 59/26; A01N 59/06

(52) U.S. Cl. 424/602; 424/687; 424/688; 424/692; 514/167

(57) ABSTRACT

A preparation of a solid calcium bolus product for preventing or treating milk fever of dairy animals is described by mixing water with calcium chloride, calcium propionate, calcium carbonate, calcium phosphate, calcium oxide, magnesium oxide, vitamin D or their combination in particle or powder form at a weight ratio of 10:100 to 45:100, introducing the mixture into moulds, and setting the moulds for 0.02 to 5 hours to convert a sludge mass into a solid bolus product. The solid calcium bolus product may be further coated with liquid molasses with fatty acid calcium salts to have a sweet molasses taste.

### BACKGROUND OF THE INVENTION

The present invention relates to the processing method of preparing a solid calcium bolus product from calcium compound(s) in particle or powder form for treating or preventing milk fever of dairy cows. The solid calcium bolus product can also be coated with a molasses liquid with fatty acid calcium salts to have a sweet molasses taste.

Milk fever or parturient paresis is a metabolic disorder of dairy cows that occurs more frequently in high-producing cows. In the disorder cases, the cows fail to absorb or mobilize enough calcium at the time of parturition to provide for the production of milk. It is associated with the sudden onset of lactation and usually occurs within 72 hours after calving. The most consistently found symptom in milk fever cases is a low blood calcium level. If treatment for such cases is not immediate and successful, there may be a danger to the cows. Treatment in all cases attempts to bring the serum or plasma calcium level to normal.

It is well known to treat milk fever by oral administrating aqueous calcium chloride solution or gel to the sick cows. But aqueous calcium chloride has a very bitter taste and is unpleasant. Therefore the sick cows must be force-treated. This may cause the calcium chloride solution to enter the lungs, which may be fatal to the cows. The gel of calcium chloride also has the same bitter taste. Another method to treat the cows from milk fever is to use capsules containing powdery calcium compound(s). But the calcium content in such capsules is relatively low and more capsules are needed. Also the calcium compounds are released too fast for the absorption in the cows. A solid bolus product with both fast and slow releasing calcium sources should be the best way to treat the cows orally for the convenience, time and taste issues. The taste of calcium chloride is needed to be limited. A number of patents have been issued for some attempts. Boehringer Ingelheim Agrovet A/S (Hellerup, Denmark) is producing a calcium bolus product, which made from both calcium chloride and calcium sulphate together under U.S. Pat. No. 5,395,622. This patent discloses that calcium chloride can not be converted into a solid calcium bolus product. Only calcium chloride with binding water and calcium sulphate together is mixed with water into a pumpable mixture, introduced into moulds and heated so that the mixture solidifies into a solid calcium bolus product, which is used to treat or prevent milk fever of dairy animals. Also the solid calcium bolus product is coated with a chemical polymer to prevent the taste issue of the product. But the coated product has a major problem to be melt at a temperature of 25 degree C. or above, which is not convenient in the applications. U.S. Pat. No. 4,338,312 discloses a method for prophylactically treating dairy cattle for parturient paresis by administrating to the cattle a 25-hydroxylated vitamin D compound and a 1.alpha.-hydroxylated vitamin D compound in combination in an amount sufficient to induce said prophylaxsis. Besides calcium, which is a major factor, vitamin D, phosphate and magnesium levels are also factors for treating or preventing the milk fever. The calcium density of the solid calcium bolus products is increased significantly compared with the calcium solution and gel. A solid calcium bolus in such as a cylinder, which could be 13 cm long and 3 cm in diameter and contain 40-45 grams of calcium, may be orally administrated to the cows by a balling gun. It takes much less time to administrate the solid calcium bolus products orally to the cows compared with administrating aqueous calcium chloride solution or gel.

### SUMMARY OF THE INVENTION

It has now been found that a solid calcium chloride itself and with nutritional ingredients of calcium phosphate, calcium propionate, calcium carbonate, calcium oxide, vitamin D, magnesium oxide or their combination may be produced from an aqueous mixture. The present invention overcomes the problems of the current calcium bolus products and provides a novel process to convert the calcium compound(s) in particle or powder form into a solid product as a calcium bolus product.

Commercial calcium chloride has three common forms of without binding water (anhydrous/CaCl.sub.2) and one molecule of calcium chloride binds with two water molecules (dihydrate/CaCl.sub.2. 2H.sub.2 O) or six water molecules (hexahydrate/CaCl.sub.2. 6H.sub.2 O) in different particle sizes or powder form. Calcium chloride in anhydrous and didydrate forms are preferred. Calcium chloride is mixed with water at 10 to 45 against 100 (calcium chloride) range on a weight basis into an uniform mass and is transferred into a mould. When calcium chloride is mixed with water at more than 18 against 100 (calcium chloride) on a weight basis, a flowable mass in a sludge liquid form is formed and may be poured into a mould. The mould may be made from paper, plastic or metal in different shapes such as a cylinder. The sludge mass forms a hard solid such as a hard solid cylinder stick after setting at room temperature for a period of time such as from 0.02 to 5 hours. In the most cases, the solidification from the mixture mass in a sludge form into a solid form needs about ½ to 2 hours. In Nielsen's U.S. Pat. No. 5,395,622, the added water amount is 52 against 100 (calcium chloride), which is higher than 45 against 100 (calcium chloride) in this invention, so there is no solid formed after setting for a period of time when there is too much water. Only the mixture of calcium chloride with binding water (one molecule of calcium chloride binds a number greater than 0 and lower than 6 water) and calcium sulphate together with water has been found to form the solid after ½ to 24 hours in U.S. Pat. No. 5,395,622. We have found that calcium chloride can be converted into a sludge mass after mixing with water at 10 to 45 against 100 (calcium chloride) range on a weight basis and into a solid form after pouring into a mould and setting for a period of time such as 1 hour. We also have found that calcium chloride can be mixed with other calcium ingredients such as calcium phosphate, calcium propionate, calcium carbonate, calcium oxide or their combination in particle or powder form with or without binding water at 0.01 to 300 against 100 (calcium chloride) on a weight basis, be mixed with water at 10 to 45 against 100 (all calcium mixture) to form a flowable mass in a sludge liquid form and be converted into a solid calcium bolus product after setting for a period of time such as from 0.02 to 5 hours. Within 10 to 45 against 100 range for water against all calcium mixture in particle or powder form, 15 to 30 against 100 range is preferred. When the sludge has a low viscosity at more than 18 against 100, the sludge is easy to be poured into a mould. When sludge has a high viscosity at less than 18 against 100, the sludge may be transferred into a mould and pressed mildly in the mould to have a product with higher density calcium and less water. The viscosity situations are the same for straight solid calcium chloride bolus product. Calcium phosphate is a better nutritional ingredient than calcium sulphate from nutritional point. Other ingredients such as magnesium oxide, vitamin D, nutritional ingredient(s), medicine(s) or their combination may be mixed with above calcium ingredient(s) and water into the solid bolus product. Calcium chloride and calcium propionate are water soluble, which may release faster in the cows. Calcium phosphate and calcium carbonate are not water soluble, which may release slower in the cows. Fast and slow calcium releases are the best for the cows to absorb calcium over the time. It is well known that phosphate, vitamin D and magnesium also contribute to treat or prevent milk fever of the cows besides calcium. We also have found when calcium chloride in anhydrous form (CaCl.sub.2) without binding water is mixed with calcium sulphate at 0.01 to 300 against 100 (calcium chloride) on a weight basis and mixed with water at 10 to 45 against 100 (the mixture of calcium chloride and calcium sulphate) to form a flowable mass, a solid calcium bolus product is formed after the flowable mass is introduced into a mould and set for a period of time from 0.02 to 5 hours. It is different from the claim 1 in Nielsen's U.S. Pat. No. 5,395,622, which requires the calcium chloride with binding water (one molecule of calcium chloride binds a number greater than 0 and lower than 6 water). Calcium chloride with or without binding water can be used in this invention. The solid calcium may be used for other nutritional applications besides for dairy cows. The solid calcium bolus product made in this invention has no melting problem after setting in a plastic bag at room temperature for 90 days.

A gel form of calcium chloride is still used in many cases now. The gel is administered orally several times immediately prior to and after calving. However this method is very unpleasant for the cows because the taste of calcium chloride is very bitter. Boehringer Ingelheim Agrovet A/S (Hellerup, Denmark) is producing the calcium bolus, which made with both calcium chloride and calcium sulphate under U.S. Pat. No. 5,395,622, coated with a chemical polymer to prevent the taste of the product. But melting problem at a temperature at 25 degree C. or above for the calcium bolus to stick onto the bolus plastic container, especially the summer, is a major issue. It is very difficult for users to get the solid calcium bolus product out of the containers and is not convenient in the application. A thin paper may be wrapped and glued onto the calcium bolus product to prevent the taste issue. We also have found a treated molasses in a liquid form may improve the taste issue. Molasses, which may be from vegetable or animal sources, has a sweet taste. Beet and cane molasses ingredients are two major molasses sources, which are the byproducts from sugar processes. Molasses is a viscous liquid ingredient, which is very hydrophilic and absorbs moisture from the air easily. Native molasses does not fit for the coating purpose for calcium bolus product.

It has been found that molasses treated with fatty acid calcium salts in a liquid form may be converted into a solid form, which has molasses taste and does not absorb moisture from the air easily. When the treated molasses in the liquid form is coated onto the calcium bolus product by immersing, spraying or brushing, then a solid coating forms after setting at room temperature for a period of time such as 2 minutes to 2 hours. Liquid molasses is mixed with a liquid oil ingredient with free fatty acid level from 25 to 100% and calcium hydroxide or calcium oxide and heated to above 80 degree C. to form a non-oily uniform liquid. When a fast agitation such a blender is used, calcium hydroxide or calcium oxide in powder form is added and mixed thoroughly with the molasses and oil. Uniform mixture helps the reaction between calcium and free fatty acids to form fatty acid calcium salts. When a slow agitation is used, calcium hydroxide or calcium oxide is mixed with water to make a 25 to 50% solution at first. Then the solution is mixed with molasses. Finally the liquid oil is added and mixed to form an uniform mass. Calcium hydroxide or calcium oxide solution can not be united with the liquid oil to form a non-oily uniform liquid. The solution of calcium hydroxide or calcium oxide is hydrophilic. The oil is hydrophobic and oily. For viscous animal fat ingredients, a mild heat may be needed to warm up into a liquid form. An oil ingredient with or without free fatty acids is normally a liquid. Animal fat is a viscous liquid because of its highly saturated level (about 50%), which becomes low viscous oil after heat. When an oil ingredient with free fatty acid level from 25 to 100% is reacted with calcium, free fatty acids and calcium form fatty acid calcium salts. Vegetable oil soap stock, which is a byproduct from vegetable oil processes, may be used for this purpose. Such as soybean soap stock has free fatty acid level about 45%, fat level 85% and moisture level less than 3%. Fatty acid calcium salts are non-oily and not water soluble, which may improve solid molasses to become more hydrophobic. Fatty acid calcium salts have a bypass function for dairy animals. The solid molasses with fatty acid calcium salts, which is formed from the above non-oily liquid when cooling to lower than 60 degree C. by setting for period of time, may be also used for other nutritional applications. Ingredients such a proteins, vitamins, phosphate and medicines may be mixed with the liquid molasses with fatty acid calcium salts and then to form a solid product after cooling down to lower than 60 degree C. Solid molasses or fat products are easy for handling, delivering, pelleting, and applying than the liquid molasses or oil products. Non-oily solid molasses with fatty acid calcium salts provides a sweet molasses taste.

Calcium chloride (especial in powder form) is very hydroscopic and water soluble, which absorbs and binds water easily. It has been found that heat is released when calcium chloride is mixed with water. Calcium chloride binds with certain amount of added water, which ranges from 10 to 45 against 100 (calcium chloride) in this invention, to form a hard solid product from a flowable mass in a liquid sludge form after setting for period of time.

There are polar and non-polar groups in the molecules of oil or fat with a free fatty acid level from 25 to 100% on a solid basis such as vegetable soap stock. The non-polar groups in the molecules bind each other with hydrophobic bonds. The polar groups (—COOH) of free fatty acids are changed into salts such as calcium salts after reacting with calcium hydroxide or calcium oxide. The calcium salts are non-oily and may be converted into a solid form, which encapsulates the non-polar parts of the oil or fat. When vegetable soap stock is mixed and reacted with calcium hydroxide or calcium oxide, the calcium salts are formed. The oily property is converted into non-oily property. A heat process is used. The moisture in the molasses and fatty acid calcium salts may be reduced by a heat process or heat and vacuum together. Then a solid product from the liquid molasses with fatty acid calcium salts is formed after cooling down to lower than 60 degree C. by setting at room temperature for a period of time or cooling air, which depends on the product weight. More weight needs longer time to cool down.

### DETAIL DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

Calcium chloride (158 grams) was mixed with water (36 grams) into a flowable liquid sludge mass. The ratio of water against calcium chloride was 22.8 to 100 (36/158). The liquid sludge mass was poured into a cylinder mould with an internal diameter of 3.4 cm. After setting at room temperature for one hour, a solid calcium chloride product was made.

### EXAMPLE 2

Calcium chloride (97 grams) was mixed with calcium phosphate (42 grams). Then water (31 grams) was mixed with the above mixture of calcium chloride and calcium phosphate into a flowable liquid sludge mass. The ratio of calcium phosphate against calcium chloride was 43 to 100 (42/97). The ratio of water against the calcium mixture was 22.3 to 100 (31/139). The liquid sludge mass was poured into a cylinder mould with an internal diameter of 3.4 cm. After setting at room temperature for two hours, a hard solid calcium product was made. A thin paper was then wrapped and glued onto the solid calcium product to prevent the product to be exposed. The solid calcium bolus product was placed in a plastic bag and set at room temperature for 60 days without any physical change.

### EXAMPLE 3

Calcium chloride (92 grams) was mixed with calcium carbonate (49 grams) and calcium propionate (11 grams). Then water (26 grams) was mixed with the above mixture of calcium chloride, calcium carbonate and calcium propionate into a flowable liquid sludge mass. The ratio of calcium carbonate and calcium propionate against calcium chloride was

65 to 100 ((49+11)/92). The ratio of water against the calcium mixture was 19.7 to 100 (26/(92+49+11)). The liquid sludge mass was poured into a cylinder mould with an internal diameter of 3.4 cm. After setting at room temperature for one and half hours, a solid calcium product was made. A thin paper was then wrapped and glued onto the solid calcium product to prevent the product to be exposed.

### EXAMPLE 4

Beet molasses (154 grams) was mixed with soybean soap stock oil (46 grams) and calcium hydroxide (7 grams). Then the mixture was mixed and heated to 130 degree C. for 25 minutes to form an uniform liquid. The solid calcium product from Example 1 was immersed into the liquid of the molasses with fatty acid calcium salts and pulled out and set at room temperature for two hours. A solid molasses coating on the solid calcium product was formed. The left of the liquid of the molasses with fatty acid calcium salts was heated and mixed for another 15 minutes and set at room temperature for three hours to form a solid product, which was then ground into particles with a coffee grinder.

What is claimed is:

1. A method of preparing a solid calcium product comprising the steps of
(a) mixing calcium chloride with calcium propionate, calcium carbonate, calcium phosphate, calcium oxide, magnesium oxide, vitamin D or their combination in particle or powder form at 0 to 300 against 100 (calcium chloride) on a weight basis to form a mixture,
(b) adding water to said mixture at 10 to 45 (water) against 100 (said mixture) on a weight basis and mixing into an uniform mass,
(c) introducing said mass into a mould,
(d) setting said mass in said mould for 0.02 to 5 hours to form the solid calcium product.

2. The method of claim 1 wherein the mould is made from paper, plastic or metal.

3. The method of claim 1 wherein an ingredient is added.

4. The method of claim 1 wherein the solid calcium product is coated with a liquid molasses with fatty acid calcium salts (claim 5) by immersing, brushing or spraying and then cooling down to lower than 60 degree C.

5. A method of preparing a liquid molasses with fatty acid calcium salts comprising mixing liquid molasses with calcium hydroxide or oxide and an oil ingredient with free fatty acid level from 25 to 100% on a solid basis and heating to above 80 degree C. to form an uniform liquid.

6. The method of claim 5 wherein the liquid is used for a coating purpose.

7. The method of claim 5 wherein an ingredient is added.

8. The method of claim 5 wherein a solid product is formed after the temperature of the liquid molasses with fatty acid calcium salts is lower than 60 degree C.

9. A method of preparing a solid calcium product comprising mixing calcium chloride in anhydrous form with calcium sulphate in particle or powder form at 0.01 to 300 against 100 (calcium chloride) on a weight basis to form a mixture, adding water to said mixture at 10 to 45 (water) against 100 (said mixture) on a weight basis and mixing into an uniform mass, introducing said mass into a mould, and setting said mass in said mould for 0.02 to 5 hours to form the solid calcium product.

10. The method of claim 9 wherein the mould is made from paper, plastic or metal.

11. The method of claim 9 wherein an ingredient is added.

12. The method of claim 9 wherein the solid calcium product is coated with a liquid molasses with fatty acid calcium salts (claim 5) by immersing, brushing or spraying and then cooling down to lower than 60 degree C.
